(19) United States
(12) Patent Application Publication
(10) Pub. No.: US 2025/0344966 A1
(43) Pub. Date: Nov. 13, 2025

(54) Accurate Ambulatory Gait Analysis with Wearable Sensors UsingTransductive Learning Inference Models

(71) Applicant: The Trustees of The Stevens Institute of Technology, Hoboken, NJ (US)

(72) Inventors: Damiano Zanotto, Jersey City, NJ (US); Ton Duong, Bloomfield, NJ (US); Huanghe Zhang, Jersey City, NJ (US)

(73) Assignee: The Trustees of The Stevens Institute of Technology, Hoboken, NJ (US)

(21) Appl. No.: 18/288,629

(22) PCT Filed: May 2, 2022
(86) PCT No.: PCT/US2022/027323

§ 371 Date: Oct. 27, 2023

Related U.S. Application Data
(60) Provisional application No. 63/182,723, filed on Apr. 30, 2021.

(51) Int. Cl. A61B 5/11; A61B 5/00; A61B 5/103; G06N 20/10

(52) U.S. Cl. CPC A61B 5/112; A61B 5/1038; A61B 5/1112; A61B 5/6807; A61B 5/7267; A61B 5/7275; G06N 20/10; A61B 2562/0219

(57) ABSTRACT

The present invention relates to a method for creating an individualized machine learning inference model. In accordance with the invention, the aforementioned method and a related system involve a motion capture device adapted to be worn by a user. The motion capture device is adapted to acquire measurements, which are used to compute a first estimate of one or more gait parameters. Next, a database is accessed that contains previously collected observations of gait data and the first estimate is compared to the previously collected observations of gait data. Finally, the subset of previously collected observations of gait data that is most informative for the particular user is identified and the individualized machine learning inference model can be developed using the identified subset of previously collected observations of gait data.

### CROSS-REFERENCE TO RELATED APPLICATION

This is an application under 35 U.S.C. § 371 of International Application No. PCT/US2022/027323 filed May 2, 2022, entitled “ACCURATE AMBULATORY GAIT ANALYSIS WITH WEARABLE SENSORS USING TRANSDUCTIVE LEARNING INFERENCE MODELS,” which claims priority to U.S. Provisional Patent Application Ser. No. 63/182,723 filed Apr. 30, 2021, the entire disclosures of both applications being incorporated herein by reference for all purposes.

### STATEMENT REGARDING FEDERALLY SPONSORED RESEARCH

This invention was made with government support under Grant Nos. IIS-1838799 and IIS-1838725 awarded by the National Science Foundation. The government has certain rights in the invention.

### FIELD OF THE INVENTION

The present invention relates to ambulatory gait analysis using foot-mounted inertial and force sensors.

### BACKGROUND OF THE INVENTION

High validity and reliability are desirable features of gait analysis instrumentation used in clinical assessments, since they make it possible to capture subtle changes in gait patterns that may indicate adverse outcomes, underlying neurological conditions, or patients' responses to an intervention. While new wearable sensing technologies have opened up a realm of new possibilities for better evaluation of patients' performance without the constraints associated with traditional laboratory instrumentation, their modest accuracy in estimating spatiotemporal and kinetic gait parameters currently limits their clinical utility.

Learning-based end-to-end inference models have been applied in recent years to estimate kinematic gait parameter using wearable sensors. These methods are typically computationally demanding and their accuracy is heavily affected by hyperparameters tuning and input features selection. Most importantly, the accuracy of these models is very sensitive to intra- and inter-subject variability, which make them less suitable for clinical applications. For this reason, current ML inference methods for wearable devices require subject-specific labelled data to train the models.

Most existing ML approaches for gait analysis rely on end-to-end models that do not take full advantage of the domain knowledge and require subject-specific labelled data to train the models. These approaches have limited practical applicability, since they cannot be applied without a reference system (i.e., gait lab equipment), which is expensive and not widely available.

### SUMMARY OF THE INVENTION

To address the problems of existing technologies, a new transductive learning framework that produces individualized inference models without the need for subject-specific labelled data was developed. This includes a new machine learning (ML) inference framework to improve accuracy, precision, and reliability of instrumented footwear for out-of-the-lab spatiotemporal gait analysis. In the proposed approach, the spatiotemporal metrics estimated with conventional data processing methods are embedded into ML inference models as domain knowledge and augmented with a subject-tailored subset of input features that substantially reduce measurement errors. By leveraging the transductive learning paradigm, the proposed framework generates personalized inference models without requiring subject-specific reference data to train the models, and therefore holds considerable potential for out-of-the lab and in-clinic gait assessments, for which laboratory equipment is often not available.

The invention is a new computational framework based on machine-learning regression, which can be used to improve accuracy and precision of wearable motion capture systems for human motion analysis, without altering the sensor hardware and low-level software/firmware. Indeed, the developed algorithms operate “downstream” relative to standard data processing algorithms for wearable devices. Thus, more accurate measurements can be obtained from the same sensors, or alternatively, a target level of accuracy can be achieved with more affordable, mid-grade sensors.

The models proposed apply ML inference algorithms (e.g., Support Vector Regression (SVV), Gaussian Process Regression (GPR), Gaussian Mixture Models (GMM), and others) to the outputs of standard data processing techniques, in order to reduce measurement errors in the biomechanical data. In these models, the outputs of conventional data processing techniques for wearable sensors are regarded as “domain knowledge” and augmented with a subject-tailored subset of features (from the wearable sensors) to reduce measurement errors. This is achieved by leveraging a previously-collected set of observations, where the outputs of a wearable system and those of reference laboratory equipment (force plates, optical motion capture, electronic walkway, or other systems, etc.) are collected simultaneously. This procedure results in more efficient (i.e., less computationally demanding and more accurate) learning from the same training data-set and sensor hardware, compared to state-of-the-art end-to-end ML models. The method also results in significantly better accuracy and precision compared with conventional data processing methods.

The application of the transductive framework allows for the extraction of the most informative subset of observations and features for any given wearer, without requiring reference data to be collected from the wearer (i.e., without the need for a measurement session in a gait laboratory). Therefore, the application of transductive learning models can train personalized (i.e., subject-tailored) inference models from a “generic” data-set of labelled data. This approach greatly increases the range of applicability of ML methods, since reference laboratory equipment is not required to train individualized models. Furthermore, because the method, unlike end-to-end ML approaches, operates on processed variables instead of raw time-series, the obtained models are computationally simple and can potentially run on embedded logic, in real-time.

The present invention relates to a method for creating an individualized machine learning inference model that involves the following steps: A user is provided with a wearable motion capture device. Then measurements are acquired using the wearable motion capture device followed by the computing of a first estimate of one or more gait parameters using the measurements. Next, a database is accessed that contains previously collected observations of gait data from the user or from other users obtained with the wearable motion capture device or from a more accurate reference device. Finally, the subset of previously collected observations of gait data (i.e., parameters) that is most informative for the particular user is identified and the individualized machine learning inference model can be developed using the identified subset of previously collected observations of gait data from the wearable motion capture device or from the reference device. In some embodiments, measurements can be taken via a system that includes at least one insole module for placement in a shoe of a user. The insole module itself may include a piezoresistive sensor, an inertial sensor, a logic unit communicatively coupled to the piezoresistive sensor and to the inertial sensor, and a transmission unit. The insole module may also interface with the database and a computing unit adapted to implement the aforementioned method.

In an embodiment, the method can be expanded to encompass the step of applying the individualized machine learning inference model to the measurements to obtain a second estimate of one or more gait parameters. The gait parameters of the first estimate can be the same parameters as those of the second estimate, or different parameters from the gait parameters of the second estimate. In some embodiments, the measurements and/or gait parameters relate to center of pressure, dynamic margin of stability, inter-limb parameters, stride length, foot-ground clearance, foot trajectory, cadence, double support time, single support time, walking or running speed, center of pressure, stride width, and margin of stability.

The method can be further expanded to encompass generating dynamic plantar pressure maps and/or center of pressure trajectories based on the measurements. The method can be further expanded to encompass the step of classifying activities of daily living based on the measurements, with or without integrating additional inputs from sensors embedded in off-the-shelf mobile devices such as a mobile phone and a wrist-worn device. Another embodiment might entail implementing the method in a mobile device having GPS in order to realize a portable navigation system. The method could also be used in conjunction with administering walking and/or balance exercises by monitoring the measurements. This could facilitate providing gait and/or balance rehabilitation to the user, either remotely or in-person. Another potential application is using the method to diagnose medical conditions affecting human gait and balance, or predicting the risk of musculoskeletal injuries.

In an embodiment, the method is performed by one or more single-board computers running a Linux distribution with a real-time kernel operating in headless mode. Such single-board computers may be configured to synchronize the measurements and write the measurements to a micro-SD card.

Various techniques can be used in conjunction with the individualized machine learning inference model, including Support Vector Regression, Gaussian Mixture Models, Gaussian Process Regression and Support Vector Machines. In another embodiment, the first estimate of one or more gait parameters is obtained by using conventional data processing techniques to obtain spatiotemporal, kinematic or kinetic gait parameters. In yet another embodiment, the individualized machine learning inference model is adapted to be implemented through a cloud service or mobile device.

The inventive concepts described above are useful for quantitative gait analysis (both in-clinic and out-of-the-lab use) and for clinical assessments, biomechanical research, and sport science. Functional gait assessments via telehealth software are also enabled, as well as longitudinal assessments of new treatments (e.g., pharmacological intervention) and natural disease history. The present invention could also potentially be extended to fall-risk mitigation/prevention in frail older adults, real-life gait monitoring and activity classification for objective physical status determination in patients and healthy individuals, and even performance analysis in runners and other athletes.

### BRIEF DESCRIPTION OF THE FIGURES

For a more complete understanding of the present invention, reference is made to the following detailed description of various representative embodiments considered in conjunction with the accompanying drawings, in which:

FIG. 1 is a schematic diagram depicting prior art footwear useful in practicing the methods of the present invention;

FIG. 2 is a flow diagram illustrating a sample implementation of the methods of the present invention; and

FIG. 3 is various views of a further shoe-based interface useful in practicing the present invention.

### DETAILED DESCRIPTION OF VARIOUS EMBODIMENTS

Embodiments will now be discussed in more detail referring to the drawings that accompany the present application. In the accompanying drawings, various embodiments are illustrated. It is to be understood, however, that these embodiments are merely illustrative of the invention, which can be embodied in various forms. In addition, the specific features of the illustrated embodiments are intended to be illustrative, and not restrictive. Further, the figures are not necessarily to scale, and some features may be exaggerated to show details of particular components with the understanding that sizes, materials and similar details shown in the figures are intended to be illustrative and not restrictive. Therefore, specific structural and functional details illustrated in the accompanying drawings are not to be interpreted as limiting, but merely as a representative basis for teaching one skilled in the art how to make and use the embodiments disclosed and illustrated herein.

Subject matter will also be described in the following text with reference to the accompanying drawings. The subject matter described hereinafter may, however, be embodied in a variety of different forms and, therefore, such subject matter should not be construed as being limited to any of the exemplary embodiments described herein. Among other things, for example, the disclosed subject matter may be embodied in the form of methods, devices, components, systems and/or combinations thereof. The following detailed description is, therefore, not intended to be taken in a limiting sense.

Throughout the Specification, terms may have nuanced meanings suggested or implied in context beyond an explicitly stated meaning. Likewise, the phrase “in one embodiment” as used herein does not necessarily refer to the same embodiment and the phrases “in another embodiment” and “other embodiments” as used herein do not necessarily refer to a different embodiment. It is intended, for example, that the disclosed subject matter includes combinations of the exemplary embodiments, in whole or in part.

In general, terminology may be understood, at least in part, from usage in context. For example, terms, such as “and,” “or,” or “and/or,” as used herein may include a variety of meanings that may depend, at least in part, upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

With the foregoing prefatory comments in mind, what follows is a detailed description of various exemplary embodiments.

The present invention was developed as an expansion of previous work, such as pending U.S. patent application Ser. No. 16/457,730 to Zanotto et al., entitled Wireless and Retrofittable In-Shoe System for Real-Time Estimation of Kinematic and Kinetic Gait Parameters (see U.S. Patent Publication 2020/0000375 and U.S. patent application Ser. No. 15/305,145 to Agrawal et al., entitled Gait Analysis Devices, Methods and Systems-see U.S. Patent Publication 2017/0055880). Both of these patent publications are incorporated herein by reference and made a part of the present disclosure for all purposes.

In the later patent publication identified above, a device comprising two insole modules and a data logger was proposed. Each insole module was wireless, having a transmission unit, as well as the ability to accurately measure kinematic and kinetic gait parameters of a user in a variety of dynamic tasks (e.g., walking, running, negotiating stairs, etc.), both outdoor and indoor. In an embodiment, all the data were collected at 500 Hz and sent wirelessly to a battery-powered single-board computer (or mobile device) running a data-logger. In an embodiment, the single-board computer fit inside a running belt that can be worn by the user or can be optionally located offboard within a 30-meter range from the user.

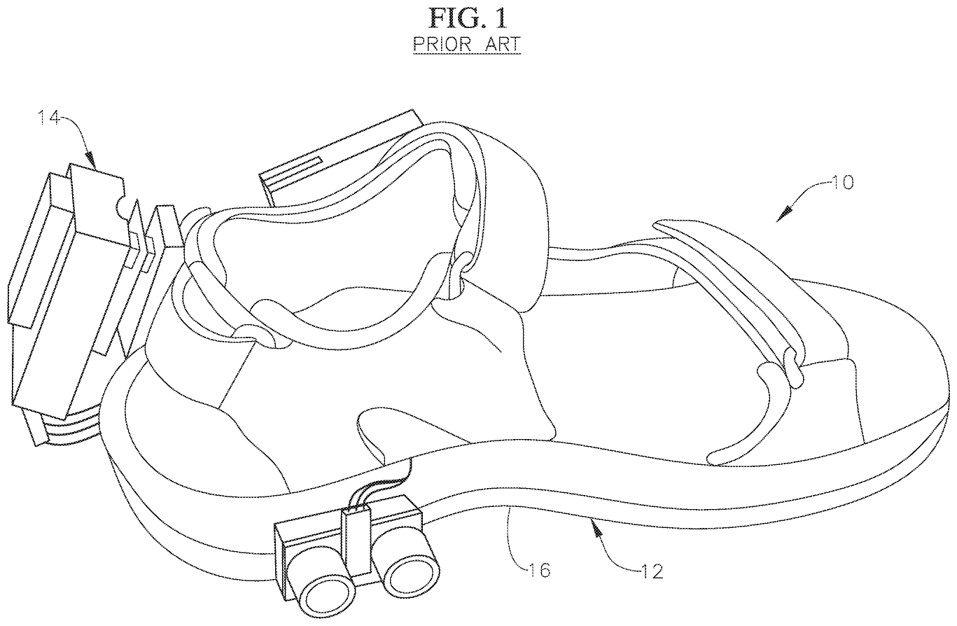

In the earlier patent publication identified above, and with reference to FIG. 1 of the present application, which corresponds to FIG. 10D of the earlier patent publication, a footwear module 10 includes a piezoresistive sensor 12, an inertial sensor 14, and a custom-made logic unit 16 (not shown). The piezoresistive sensor 12 and logic unit 16 are embedded, inlaid or otherwise attached to the sole of the footwear module 10. The pressure sensors (i.e., the piezoresistive sensors 12) can be located, for instance, underneath the calcaneous, the lateral arch, the head of the first, third and fifth metatarsals, the hallux, and the toes, while the inertial sensor 14 can be located, for instance, along the midline of the foot.

As described in the later of the patent publications identified above, the logic unit 16 may include a microcontroller interfaced with the multi-cell pressure sensor through an eight-channel multiplexer, while communicating with the inertial sensor 14 through a serial connection. In an embodiment, all the data are sampled at 500 Hz and sent through UDP over WLAN to the single-board computer by means of a Wi-Fi module. The logic unit 16, which can be housed in a plastic enclosure, is powered by, for instance, a small 400 mAh Li-po battery through a step-up voltage regulator.

The single-board computer is adapted to run a Linux distribution with a real-time kernel operating in headless mode. A miniature Wi-Fi router can be connected to the computer, serving as an access point. In use, for example, the computer synchronizes the data incoming from the footwear module 10 and writes them to a micro-SD card. The same data can also be streamed at a lower sample rate (50 Hz) to an easy-to-use user interface running on the user's laptop or mobile phone, whereby the interface allows the user to control the system remotely and to visualize measured data.

With particular reference to the present invention, it offers a novel transductive learning framework to improve validity and reliability of wearable devices, and its validation with a particular subclass of wearable device (instrumented insoles). To do so, ML inference models (SVR, GPR, and others) are applied to spatiotemporal and kinetic gait parameters. The envisioned data analysis “pipeline” comprises the following steps:
1) acquire data using a wearable motion capture device;
2) apply standard data processing techniques to extract “conventional” estimates of a target spatiotemporal, kinematic, or kinetic gait parameters;
3) use the conventional estimates of the target gait parameter along with other sensor features to identify the best subset of previously collected observations (from an existing database) that is the most informative for the current wearer, and develop an individualized ML inference model (i.e., transductive inference);
4) apply the individualized model to the conventional data to minimize measurement errors in the selected gait parameter; and
5) repeat steps 2-3-4 for all gait parameters of interest.

Once trained, the ML model runs on embedded logic or, alternatively, raw data can be processed via a cloud service or a mobile device.

Insoles instrumented with inertial (IMU) and force (FSR) sensors capture raw gait data (time-series) from the wearer in real-life or controlled environments. These sensors, which are known in the art, are discussed, for instance, in the aforementioned patent publications which have been incorporated by reference hereinabove. Data are stored in the onboard data-logger at a selectable rate 333-500 Hz and a phone App is used to control the recording process. Raw data from L/R insoles are synchronized within millisecond accuracy using BLE “connect events” and “Reference Broadcast Synchronization.” External instrumentation (e.g., lab equipment or wearable sensors) can also be synchronized through the phone app or an auxiliary wireless “sync box.” Machine-learning (ML) inference models are applied to data extracted from the insoles, to compute spatiotemporal and kinetic gait parameters with high accuracy and precision.

The ML models are trained using a dataset of previously-collected observations (i.e., data from the insoles and synchronized data from laboratory equipment). The transductive learning framework is applied to automatically select the most informative subset of previously collected observations to tune the models to the current user, without the need for test subject-specific labelled data. The ML models adjust the outputs of conventional data processing techniques for wearable sensors, resulting in significantly improved accuracy and precision. Summary data can then be presented to the user and/or the clinician through a graphical interface.

The ML models can run either locally or remotely, in a cloud service. As more observations are collected and transmitted to the cloud server along with reference data, the ML models can be refined.

Additionally, the present invention also includes a framework to precisely synchronize wearable sensors with external equipment. While this method allows for the efficient training of ML models, it is not limited to specific setups delineated herein. In fact, the same approach can be extended to other wearable sensors and other types of laboratory equipment, thereby allowing a user (e.g., a manufacturer) to conduct rigorous sensor validation.

By using conventional data processing methods for wearables and ML regression models, the present invention can help improve validity and reliability of wearable motion capture systems. This results in computationally simpler models that can run on embedded logic and are relatively robust to inter- and intra-subject differences, unlike end-to-end ML inference models

The use of transductive learning to obtain subject-tailored inference models from generic datasets (i.e., observations previously collected from other subjects) has the potential to improve validity, reliability, and range of applicability of current wearable technologies for gait analysis, and can greatly extend the practical applicability of ML methods in such field.

Furthermore, the framework for wireless synchronization will allow manufacturers, developers, and clinical researchers to validate new and existing wearable devices by concurrently measuring human motion using the wearable device under investigation and ground-truth gait-lab instruments.

The proposed method includes an additional step, or, in other words: the application of ML-inference models. This step can substantially improve accuracy and therefore is greatly convenient in all applications for which high validity and reliability are critical.

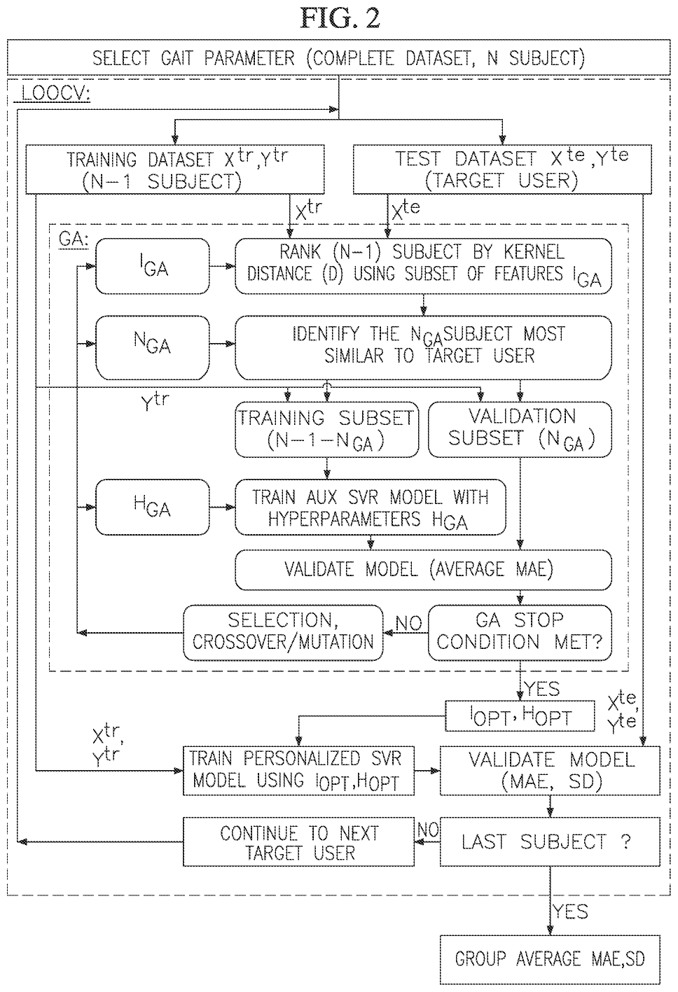

The proposed inference framework is illustrated in FIG. 2. In line with the transductive inference paradigm, the locations of the test samples in the input feature space are exploited to improve model predictions. To this end, for each target user (i.e., for each iteration of the leave-one-out cross validation (LOOCV) loop), the algorithm produces an individualized SVR model by leveraging the input feature vector X^{te} extracted from the target user. A detailed description of the steps involved in the proposed method is reported hereinbelow.

### Kernel Distance as Similarity Measure

Given a gait parameter of interest and a target user, the procedure first identifies the subset of individuals that most closely resemble the target user. These individuals will form the hold-out validation subset for the feature selection step described in the “Feature Selection and Hyperparameters Tuning” section below. To this end, the generalized distances between the training dataset and the test dataset in the feature space are computed as:

$${d}_{i , j} = ‖ ϕ ( {X}_{i}^{tr} ( I ) ) ) - ϕ ( {X}_{j}^{te} ( I ) ) ‖ = \sqrt{{K}_{i , i}^{tr} ( I ) - 2 {K}_{i , j} + {K}_{j , j}^{te}} \qquad ( l )$$

di, j is the generalized distance characterizing the degree of similarity between the i-th training instance and the j-th test instance. Vector I contains the indices of the selected input features, which are measured by the instrumented footwear. X^{tr} (I) and X^{te} (I) are the vectors of the selected input features corresponding to the training dataset and the target user, respectively. ϕ(·) represents the feature mapping function, which maps the selected subset of input features X(I) into the high-dimensional feature space.

$${K}_{i , i}^{tr} and {K}_{j , j}^{te}$$

are entries of the kernel matrices corresponding to the training dataset and the test dataset. K is the kernel matrix defined as

$$K = ( \begin{matrix}k ( {X}_{1}^{tr} ( I ) , {X}_{1}^{te} ( I ) ) & ⋯ & k ( {X}_{1}^{tr} ( I ) , {X}_{M}^{te} ( I ) ) \\ ⋮ & ⋱ & ⋮ \\ k ( {X}_{L}^{tr} ( I ) , {X}_{l}^{te} ( I ) ) & … & k ( {X}_{L}^{tr} ( I ) , {X}_{M}^{te} ( I ) )\end{matrix} ) , \qquad ( 2 )$$

where k (·) is the kernel function, and L, M are the total number of instances in the training and the test dataset, respectively. The normalized distance D₁ between the l-th
individual in the training dataset and the target user is given by

$${D}_{l} = \frac{{\sum }_{i = 1}^{{N}_{s , l}} {\sum }_{j = 1}^{M} {d}_{i , j}}{{N}_{s , l}} \qquad ( 3 )$$

where N_{s,1} is the total number of training instances in the l-th subject's dataset.

### Feature Selection and Hyperparameter Tuning

As shown in FIG. 2, an efficient heuristic optimization method based on Genetic Algorithm (GA) determines the best subset of input features Iₒₚₜ and the best SVR hyperparameters Hₒₚₜ for the target user. The GA operates through a nested cross-validation loop, wherein the N_{GA} individuals that are the most similar to the target user in terms of normalized distances (3) form the hold-out validation subset, and the remaining (N-1-N_{GA}) individuals form the training subset for an auxiliary SVR model. The number of individuals in the validation subset N_{GA}, the input feature subset I_{GA}, and the SVR hyperparameters H_{GA} are the optimization variables. For each iteration of the GA, model performance (as quantified by the mean absolute error, MAE) is evaluated for each of the N_{GA} individuals in the validation subset, and the average MAE is used as the cost function for the GA. This procedure is repeated until a stopping criterion is met.

In summary, because Iₒₚₜ and Hₒₚₜ maximize model accuracy on the group of individuals in the training dataset that most closely resemble the target user, they are regarded as the optimal parameters to train an individualized SVR model based on the training dataset (X^{tr} (Iₒₚₜ), Y^{tr}) consisting of N-1 individuals, by following the SVR procedure discussed in the “Support Vector Regression” section below. It is worth noting that, since model training is performed on the largest available dataset (as opposed to the Nopt most similar individuals), the resulting SVR model is less prone to overfitting.

### Feature Extraction

The set of candidate input features include the following variables:
1) Spatiotemporal Gait Parameters: The conventional procedure to estimate spatiotemporal gait parameters using instrumented footwear starts from the determination of gait events. The timing of the initial contacts (IC) events is computed based on underfoot FSRs. Toe-off (TO) events are detected using the change of sign in the angular velocity signal after the foot-flat event. This was motivated by preliminary tests indicating that FSRs could not accurately capture TO events in certain populations of older adults. Stride time (ST) is defined as the time interval between two consecutive ICs of the same foot. Swing time (SW) is computed as the time interval between the TO of the current stride and the IC of the same foot's next stride, and SW % is determined as the ratio between SW and the corresponding ST. Double support time (DS) is calculated as the time interval between the IC of the contralateral foot and the TO of the ipsilateral foot. FSRs are used to determine foot-flat (FF) periods. Foot displacement over consecutive FF periods is computed by double integration of the gravity-corrected accelerometric signals, along with the implementations of zero velocity update (ZUPT) and velocity drift compensation (VDC). The integration interval between consecutive FF periods is delimited by the time instant with the lowest acceleration magnitude within each FF period. Stride length (SL) is calculated as the L2-norm of the displacement vector, and stride velocity (SV) is defined as the ratio between SL and the corresponding ST.
2) Inertial Features: Inertial features are computed between two consecutive FF periods of the same foot. They include the L2-norm of the foot acceleration (|α|), as well as the root mean square (rms), maximum (max), sum, energy, and sample entropy of the vertical (a_{z}), anteroposterior (aₓ) and mediolateral (a_{y}) projections of the foot acceleration.
3) Anthropometric Features: Anthropometric attributes include height, weight, body mass index (BMI), shoe size, age, gender, and type of walking assistance (i.e., walker/cane, or no assistive device). Dummy coding was used to include categorical features in the SVR models.

### Support Vector Regression (SVR)

The goal of the SVR models is to reduce measurement errors in the gait parameters obtained with the conventional data processing techniques described in the “Feature Extraction” section above. SVR was selected over neural networks because the former has superior generalization accuracy and global optimization properties. SVR models estimate a gait parameter at the i-th stride as

$${\hat{Y}}_{i}^{tr} = f ( {{X}_{i}^{tr} ( I )}_{,} β ) = {β}^{T} ϕ ( {X}_{i}^{tr} ( I ) ) \qquad ( 4 )$$

$${Y}_{i}^{tr}$$

is the reference value of the gait parameter at the i-th stride measured with the gold-standard equipment and

$${\hat{Y}}_{i}^{tr}$$

is the corresponding SVK estimate.

$${X}_{i}^{tr} ( I )$$

is the vector or input features (restricted to the subset of features I), measured over the same stride. The weights vector β is determined by numerically solving the constrained convex optimization problem

$$min \frac{{‖ β ‖}^{2}}{2} + C {\sum }_{i = 1}^{L} ( {ξ}_{i} + {ξ}_{i}^{*} ) \qquad ( 5 )$$

$$Subject to {Y}_{i}^{tr} - {β}^{T} ϕ ( {X}_{i}^{tr} ( I ) ) \leq ε + {ξ}_{i}^{*} ,$$

$$i = 1 , … , L$$

$${β}^{T} ϕ ( {X}_{i}^{tr} ( I ) - {Y}_{i}^{tr} ) \leq ε + {ξ}_{i} , I = 1 , … , L \qquad ( 6 )$$

$${ξ}_{i} , {ξ}_{i}^{*} > 0 , i = 1 , … , L$$

where ε and C are the SVR hyperparameters. The former is a user-defined tolerance, the latter determines the trade-off between the flatness of f(X(I), β) and the extent to which deviations larger than & are penalized. ξ and ξ* are slack variables, bounding regression errors that are tolerated. Gaussian radial basis function (RBF) was selected as the type of kernel function, and the sets of candidate values for the hyperparameters were restricted to the following: C∈[1 2 5 10 100], ε∈[0.1 0.2 0.5 0.8 1 1.2 1.5 2 2.5 3]

### Statistical Analysis

To highlight the advantages of the proposed transductive models in estimating stride-by-stride spatiotemporal gait metrics, relative to the conventional data processing procedure and relative to previous work, the validity and reliability of the following methods was compared:
1) Conventional Method (CONV): This is the conventional data processing procedure described in the “Spatiotemporal Gait Parameters” section above. This method, unlike the following ones, does not rely on inference models and therefore it does not require training. The CONV method was included in the analysis to verify whether the complexity of SVR models is well-justified by improvements in accuracy and precision.
2) Subject-Specific SVR Models (SS): With the SS method, SVR models are trained independently for each subject, using that subject's labelled data. For a given set of input features, this method yields the best performance that can be obtained using SVR. However, since subject-specific labelled data are required for each individual, the method has limited practical applicability. Each model is evaluated using 10-fold cross-validation. Within each fold, feature selection and hyperparameters optimization is achieved using GA, via a nested 9-fold cross-validation procedure wherein the cost function is the average MAE across the 9 folds.
3) Generic SVR Models (GN): This method generates one-size-fits-all models using LOOCV. Within each training dataset (i.e., data from (N-1) individuals), feature selection and hyperparameters optimization is achieved using GA, via a nested 10-fold cross-validation procedure similar to the one used for SS, in which the cost function is the average MAE across the 10 folds.
4) Transductive SVR Models (TR): The present invention involves this method. It generates individualized models using an optimized subset of labelled data taken from other individuals, as described in the “Kernel Distance as Similarity Measure” section and “Feature Selection and Hyperparameter Tuning” section.

For each gait parameter, the interrater reliability of each data processing method was assessed relative to the ground-truth system using intraclass correlation coefficients (ICC), by employing a single-measurement, absolute agreement, two-way mixed-effects model. Point estimates of the ICC were rated as poor (<0.5), moderate (0.5 to 0.75), good (0.75 to 0.9), and excellent (0.9 to 1).

Validity of the data processing methods was evaluated by calculating the MAE and the standard deviation of the errors (SD) for each gait parameter relative to the ground-truth values. MAE and SD were regarded as metrics of accuracy and precision, respectively, since they are widely used in the validation of wearable sensors. Repeated-measures ANOVA with processing method (CONV, SS, GN, TR) were applied as within-subject factor, walking condition (non-assisted vs. assisted walking) as between-subject factor, and MAE and SD of the five gait parameters as dependent variables. Study participants who used a walker or a cane to perform the 6MWT were included in the assisted walking group. Mauchly's test was applied to check sphericity, and the Huynh-Feldt correction was applied if Mauchly's test indicated that the assumption of sphericity had been violated. When significant (α<0.05) effects were identified, post-hoc comparisons using the Bonferroni-Holm correction were applied as appropriate.

The level of accuracy of inertial sensors for ambulatory gait analysis typically degrades when those sensors are applied to clinical populations with motor deficits. Thus, Spearman's correlation analysis was carried out separately for each gait parameter to assess the effect of gait and balance impairment on the accuracy of the four data processing methods. The Timed Up and Go (TUG) clinical score was selected as a surrogate metric for gait and balance impairment, and percentage MAE values were regarded as representative of model accuracy, to account for inter-subject biometric differences. The strength of the correlation was interpreted as negligible (|ρ|<0.3), low (0.3≤|ρ|<0.5), moderate (0.5≤|ρ|<0.7), high (0.7≤|ρ|<0.9), or very high (0.9≤|ρ|<1.0). All statistical analysis was carried out in SPSS v28 (IBM Corporation, Armonk, NY).

### Feature Analysis

For each gait parameter, the permutation feature importance PFIl,i was determined, which yields the importance of the l-th input feature as related to the measurement errors of the TR model tailored to the i-th target user:

$$PFIl , i = \frac{{MAE}_{l , i}^{perm}}{{MAE}_{i}^{orig}} \qquad ( 7 )$$

$${MAE}_{i}^{orig}$$

is the original MAE of the i-th TR model for a given gait parameter, while

$${MAE}_{l , i}^{perm}$$

is the MAE obtained by randomly permuting the l-th input feature within X^{te}, and feeding the altered input vector to the same TR model. The PFI_{l,i} values are averaged across all subjects for whom the l-th input feature is included in Iₒₚₜ, resulting in a single scalar metric per each gait parameter. Because this procedure would not be applicable to the anthropometric features (since they remain the same for any given target user), the following modified equation was used in place of (7) for all anthropometric features

$$P {F}^{⋁} {I}_{l} , i = \frac{1}{{N}_{l}} \frac{{\sum }_{n = 1}^{{N}_{l}} {MAE}_{l , i}^{n}}{{MAE}_{i}^{orig}} \qquad ( 8 )$$

where N₁ is the number of possible values that the l-th feature can take (e.g., if 1 indicates sex, then N₁=2) and

$${MAE}_{l , i}^{n}$$

was computed by setting the l-th feature in X^{te} to its n-th value.

### Results

A total of 9253 strides were collected simultaneously by the instrumented footwear and by the reference system. The number of strides per test subject varied from 42 to 159 (97.4±24.6 mean and SD), depending on their velocity during the 6MWT. SL varied from 11.13 to 164.20 cm (83.83±25.93 cm), SV varied from 10.29 to 151.60 cm/s (70.40-26.73 cm/s), ST varied from 0.74 to 2.41 s (1.24±0.22 s), SW varied from 0.08 to 0.89 s (0.39±0.08 s), and DS varied from 0.05 to 0.93 s (0.23±0.08 s). Training and testing of the inference models were conducted on a 4 GHZ Intel® Core™ i7-6700K using MATLAB (The Mathworks Inc., Natick, MA). In the TR models, N_{GA} ranged from 1 to 10 test subjects. For each each gait parameter, it took approximately 180 minutes, 40 minutes, and 8 minutes to train GN, TR, and SS models, respectively.

### Interrater Reliability

Intraclass correlation coefficients are reported in Table II. In general, SVR models showed superior equivalence with the gold-standard equipment compared to the CONV method. However, differences in equivalence between CONV and SVRmethods were significant only in SW and DS, for the TR and SS models (as indicated by the confidence intervals (CIs) in Table II). The interrater reliability of SVR models slightly degraded when test subject-specific training data were not available, and this effect was more marked for GN models than it was for TR models. Importantly, TR models demonstrated better stability (i.e., smaller CIs) than GN models.

TABLE II
INTRACLASS CORRELATION COEFFICIENTS FOR
THE FOUR PROCESSING METHODS. VALUES
BETWEEN PARENTHESES INDICATE 95% CI

| | CONV | GN | TR | SS |
|---|---|---|---|---|
| SL | .98 | .99 | 1.00 | 1.00 |
| | (.86-.99) | (.98-.99) | (.99-1.00) | (1.00-1.00) |
| SV | .99 | .99 | 1.00 | 1.00 |
| | (.93-1.00) | (.99-.99) | (1.00-1.00) | (1.00-1.00) |
| ST | 1.00 | 1.00 | 1.00 | 1.00 |
| | (1.00-1.00) | (1.00-1.00) | (1.00-1.00) | (1.00-1.00) |
| SW | .84 | .90 | .94 | 1.00 |
| | (.77-.89) | (.86-.93) | (.90-.96) | (1,00-1.00) |
| DS | .89 | .94 | .96 | 1.00 |
| | (.84-.93) | (.91-.96) | (.94-.98) | (1.00-1.00) |

TABLE III
ACCURACY (MAE) AND PRECISION (SD) OF THE ESTIMATED
GAIT PARAMETERS (POOLED DATA, INCLUDING BOTH
ASSISTED AND NON-ASSISTED WALKING CONDITIONS)

| | CONV | | GN | | TR | | SS | |
|---|---|---|---|---|---|---|---|---|
| | MAE | SD | MAE | SD | MAE | SD | MAE | SD |
| SL [cm] | 5.87 | 5.90 | 4.01 | 3.58 | 3.36 | 3.80 | 2.60 | 3.53 |
| SV [cm/s] | 4.81 | 4.80 | 3.38 | 2.97 | 2.79 | 3.14 | 2.18 | 2.96 |
| ST [ms] | 16.66 | 24.04 | 18.74 | 24.22 | 17.31 | 24.17 | 18.15 | 25.39 |
| SW [ms] | 31.58 | 24.73 | 25.11 | 23.07 | 20.99 | 22.50 | 14.46 | 20.33 |
| DS [ms] | 32.60 | 26.69 | 26.00 | 22.50 | 21.63 | 22.64 | 15.21 | 21.01 |

### Validity

The MAE average across all test subjects are reported in Table III, along with their SD. Results of the repeated-measures ANOVA are shown in Table IV and post-hoc analyses are reported in Table V.

TABLE IV
p-VALUES OF THE TWO-WAY REPEATED-MEASURES
ANOVA (PM = PROCESSING METHOD, WC =
WALKING CONDITION)

| | MAE | | | SD | | |
|---|---|---|---|---|---|---|
| | PM | WC | PM*WC | PM | WC | PM*WC |
| SL | <0.001 | ns | ns | <0.001 | ns | ns |
| SV | <0.001 | ns | ns | <0.001 | ns | ns |
| ST | <0.001 | <0.001 | ns | <0.05 | <0.001 | ns |
| SW | <0.001 | <0.001 | <0.05 | <0.001 | <0.001 | <0.001 |
| DS | <0.001 | <0.001 | <0.05 | <0.001 | <0.001 | <0.01 |

TABLE V
Adjusted p-Values of the Post-Hoc Analyses Following One-Way Repeated
Measures ANOVA. The Bonferroni-Holm Method Was Used to Adjust for
Multiple Pairwise Comparisons Across the Processing Methods

| | CONV/GN | | CONV/TR | | CONV/SS | |
|---|---|---|---|---|---|---|
| | MAE | SD | MAE | SD | MAE | SD |
| Pooled Data | | | | | | |
| SL | <0.001 | <0.001 | <0.001 | <0.001 | <0.001 | <0.001 |
| SV | <0.001 | <0.001 | <0.001 | <0.001 | <0.001 | <0.001 |
| ST | <0.001 | ns | ns | ns | <0.001 | ns |
| Non-Assisted | | | | | | |
| SW | <0.01 | ns | <0.001 | ns | <0.001 | ns |
| DS | <0.001 | ns | <0.001 | ns | <0.001 | ns |
| Assisted | | | | | | |
| SW | Ns | ns | <0.001 | <0.01 | <0.001 | <0.001 |
| DS | Ns | <0.01 | <0.001 | <0.01 | <0.001 | <0.001 |

| | GN/TR | | GN/SS | | TR/SS | |
|---|---|---|---|---|---|---|
| | MAE | SD | MAE | SD | MAE | SD |
| Pooled Data | | | | | | |
| SL | <0.001 | <0.01 | <0.001 | ns | <0.001 | Ns |
| SV | <0.001 | <0.001 | <0.001 | ns | <0.001 | ns |
| ST | <0.001 | ns | Ns | ns | ns | ns |
| Non-Assisted | | | | | | |
| SW | <0.01 | ns | <0.001 | ns | <0.05 | ns |
| DS | ns | ns | <0.001 | ns | <0.05 | ns |
| Assisted | | | | | | |
| SW | <0.05 | ns | <0.001 | <0.001 | <0.05 | <0.01 |
| DS | <0.05 | ns | <0.001 | <0.05 | <0.05 | <0.05 |

TABLE VI
SPEARMAN'S ρ AND CORRESPONDING
p-VALUES BETWEEN TUG AND THE PERCENT
MAE OF GAIT PARAMETERS, FOR EACH
DATA PROCESSING METHOD

| MAE | TUG | | | |
|---|---|---|---|---|
| % | CONV | GN | TR | SS |
| SL | 0.478 (<0.001) | 0.687 (<0.001) | 0.583 (<0.001) | 0.640 (<0.001) |
| SV | 0.517 (<0.001) | 0.711 (<0.001) | 0.579 (<0.001) | 0.676 (<0.001) |
| ST | 0.647 (<0.001) | 0.630 (<0.001) | 0.606 (<0.001) | 0.653 (<0.001) |
| SW | 0.295 (<0.01) | 0.615 (<0.001) | 0.543 (<0.001) | 0.668 (<0.001) |
| DS | ns | 0.321 (<0.01) | 0.277 (<0.01) | 0.461 (<0.001) |

The processing method significantly affected the accuracy of SL and SV. MAE values in these gait metrics were significantly smaller when using all SVR models compared to the CONV method (Table V). SS models outperformed both GN and TR models in these two gait metrics, indicating the advantages of exploiting subject-specific labelled data, when they are available. On the other hand, it is worth noting that TR models yielded significantly smaller MAE than GN models, despite relying on the same set of available labelled observations (i.e., non subject-specific).

The processing method also affected the MAE of ST. The CONV method yielded the most accurate results in ST (see Table III), confirming the validity of conventional approaches based on FSRs to estimate this temporal parameter of gait. The CONV method outperformed both SS and GN methods (but not TR) in terms of accuracy. However, the increase in MAE between CONV and GN and SSwas very modest (on average, less than 2.1 ms, which in this sample corresponded to only 0.09_0.3% of the stride time), thereby indicating that all methods yielded excellent validity in estimating ST. Regardless of the processing method, errors in ST were larger for test subjects who walked with a mobility aid. It is presently postulated that walking with a mobility aid augments an individual's forward lean, which in turn negatively impacts the accuracy with which IC events are detected, by altering the foot loading patterns upon which IC detection algorithms are based. In terms of gait phase variables (SW and DS), the accuracy of the processing methods depended on whether test subjects walked with or without a mobility aid, as indicated by the significant interactions (see Table IV). Separate one-way repeated-measures ANOVA (see Table V) indicated that the TR and SS methods significantly outperformed the CONV method in both SW and DS for test subjects requiring a walking aid, but the improvement was only marginal for the GN method. For the non-assisted walking condition, instead, results replicated those obtained for the MAE of SL and SV (i.e., all SVR models outperformed the CONV method). Unlike ST, gait phase metrics require the detection of both IC and TO events. The difference in the performance of the CONV methods between ST and gait phase metrics may be explained by noting that capturing TO events is far more challenging than detecting IC when using foot-attached sensors, especially with elderly and impaired gait.

The analysis of the error variability (SD) for SL and SV revealed a significant effect of the processing method, with all the SVR models significantly outperforming the CONV method (see Table V). The SVR models for SL and SV performed similarly in terms of precision, except for the TR method, which resulted in significantly worse precision compared to the GN method. This result may be explained with the type of cost function chosen for the GA optimizer, which only accounted for accuracy. Similar to the MAE, the precision of ST was negatively affected by the use of a walking aid, irrespective of the processing method. A significant effect of the processing method on the precision of ST was found, but the post-hoc analysis did not reveal significant differences among CONV, GN, TR and SS, suggesting that this effect was marginal. Additionally, the precision of the processing methods in determining SW and DS depended on the walking condition. For test subjects walking without a mobility aid, no significant differences in precision were found across the processing methods in these two gait metrics. For those who walked with a mobility aid, however, all SVR models produced an improvement in precision relative to the CONV method (with SS performing better than TR and GN), even though the improvement was not significant for the GN method in estimating SW. In summary, for spatial gait parameters (SL, SV), all inference models consistently outperformed the CONV method, both in terms of accuracy and precision, regardless of the walking condition. This was also the case for the gait phase variables (SW and DS), except that significant improvements in precision were not found when applying SVR models to highly functioning test subjects who did not use a mobility aid. These results clearly indicate that when subject-specific labelled data are not available for SL, SV, SW, and DS, the TR method should be preferred to the CONV method and to one-size-fits-all (GN) models. On the other hand, no evidence was found that the application of SVR models may further improve the validity of ST beyond the high levels of accuracy afforded by the CONV method.

### Effects of Gait Impairment on Validity

The results of the bivariate correlation analysis are summarized in Table VI. Except for DS estimated using the CONV method, the percentage MAE of all gait parameters, regardless of the data processing method, were positively associated with the TUG scores (i.e., the longer the time required to complete the TUG, the larger the measurement errors). This suggests that the more severe the gait impairment is, the more challenging it is to extract accurate spatiotemporal gait parameters using instrumented footwear. Nonetheless, all correlation coefficients were low or moderate.

### Feature Analysis

Table VII shows the number of input features selected by the GA optimizer for the GN and TR models. Compared to the GN method, the number of optimal input features for the TR method was consistently smaller across all gait metrics, indicating simpler and more efficient inference models. The PFI values for the TR models are reported in Table VIII. As expected, the accuracy of the TR models was mainly affected by the estimations obtained with the conventional method (indicated as pCONV). The second most relevant feature for SL and SV was sum(ax), which is an indirect measure of the amount of velocity drift error accumulated after each stride. Indeed, this value is exactly what the VDC method exploits to cancel out the effects of velocity drift. It is presently conjectured that this input feature allows the inference models to compensate for the residual effects of velocity drift after applying VDC, thereby affecting the accuracy of both SV and SL.

TABLE VII
MEAN AND STANDARD DEVIATION (SD) OF THE NUMBER OF FEATURES
SELECTED BY THE GA OPTIMIZER FOR THE GN AND TR MODELS

| | SL | | SV | | ST | | SW | | DS | |
|---|---|---|---|---|---|---|---|---|---|---|
| | GN | TR | GN | TR | GN | TR | GN | TR | GN | TR |
| MEAN | 23.8 | 15.6 | 23.7 | 15.3 | 18.7 | 11.2 | 22.6 | 14.0 | 23.5 | 14.7 |
| SD | 1.4 | 2.9 | 1.4 | 2.9 | 1.7 | 2.5 | 1.4 | 2.6 | 1.7 | 3.0 |

TABLE VIII
PERMUTATION FEATURE IMPORTANCE FOR TR MODELS.
THE TWO MOST IMPORTANT FEATURES FOR EACH GAIT
PARAMETER ARE MARKED IN BOLD FONT. p^{CONV}
INDICATES THE CORRESPONDING GAIT PARAMETER
(I.E., SL, SV, ST, SW OR DS) OBTAINED WITH
THE CONV METHOD

| Features | SL | SV | ST | SW | DS |
|---|---|---|---|---|---|
| p^{CONV} | 2.48 | 2.77 | 6.26 | 2.41 | 1.74 |
| \|a\| | 1.08 | 1.12 | 1.05 | 1.06 | 1.07 |
| ST | 1.05 | 1.03 | — | 1.04 | 1.06 |
| SW % | 1.09 | 1.15 | 1.02 | — | 1.09 |
| rms(aₓ) | 1.12 | 1.08 | 1.06 | 1.22 | 1.08 |
| rms(a_{y}) | 1.07 | 1.05 | 1.12 | 1.11 | 1.11 |
| rms(a_{z}) | 1.16 | 1.20 | 1.07 | 1.08 | 1.19 |
| max(aₓ) | 1.00 | 1.02 | 1.01 | 1.03 | 1.01 |
| max(a_{y}) | 1.01 | 1.02 | 1.01 | 1.03 | 1.02 |
| max(a_{z}) | 1.01 | 1.01 | 1.00 | 1.04 | 1.03 |
| min(aₓ) | 1.01 | 1.01 | 1.01 | 1.00 | 1.01 |
| min(a_{y}) | 1.01 | 1.03 | 1.01 | 1.02 | 1.00 |
| min(a_{z}) | 1.00 | 1.01 | 1.00 | 1.01 | 1.01 |
| sum(aₓ) | 1.23 | 1.23 | 1.01 | 1.02 | 1.02 |
| sum(a_{y}) | 1.02 | 1.03 | 1.00 | 1.03 | 1.01 |
| sum(a_{z}) | 1.21 | 1.14 | 1.01 | 1.04 | 1.00 |
| energy(aₓ) | 1.11 | 1.17 | 1.04 | 1.11 | 1.10 |
| energy(a_{y}) | 1.09 | 1.08 | 1.11 | 1.10 | 1.09 |
| energy(a_{z}) | 1.14 | 1.10 | 1.02 | 1.13 | 1.10 |
| entropy(aₓ) | 1.01 | 1.03 | 1.00 | 1.02 | 1.00 |
| entropy(a_{y}) | 1.02 | 1.03 | 1.00 | 1.03 | 1.01 |
| entropy(a_{z}) | 1.05 | 1.05 | 1.00 | 1.05 | 1.04 |
| height | 1.09 | 1.04 | 1.00 | 1.00 | 1.11 |
| weight | 1.14 | 1.11 | 1.03 | 1.36 | 1.40 |
| BMI | 1.10 | 1.06 | 1.04 | 1.19 | 1.14 |
| shoe size | 1.14 | 1.15 | 1.06 | 1.21 | 1.30 |
| age | 1.08 | 1.04 | 1.01 | 1.05 | 1.05 |
| gender | 1.13 | 1.11 | 1.02 | 1.13 | 1.21 |
| assistance | 1.12 | 1.08 | 1.05 | 1.15 | 1.19 |

It is worth noting that, for ST, the PFI of pCONV was approximately six times larger than any other input features. This provides strong evidence that inference models using this candidate set of input features might not be as effective in correcting the already modest measurement errors in ST produced by the CONV method. For SW and DS, the second most important feature was the test subject's weight. Because the magnitude of the propulsive forces at TO is associated with an individual's weight, this parameter may correct the accuracy with which TO events (and therefore SW and DS) are detected.

### Support Vector Regression

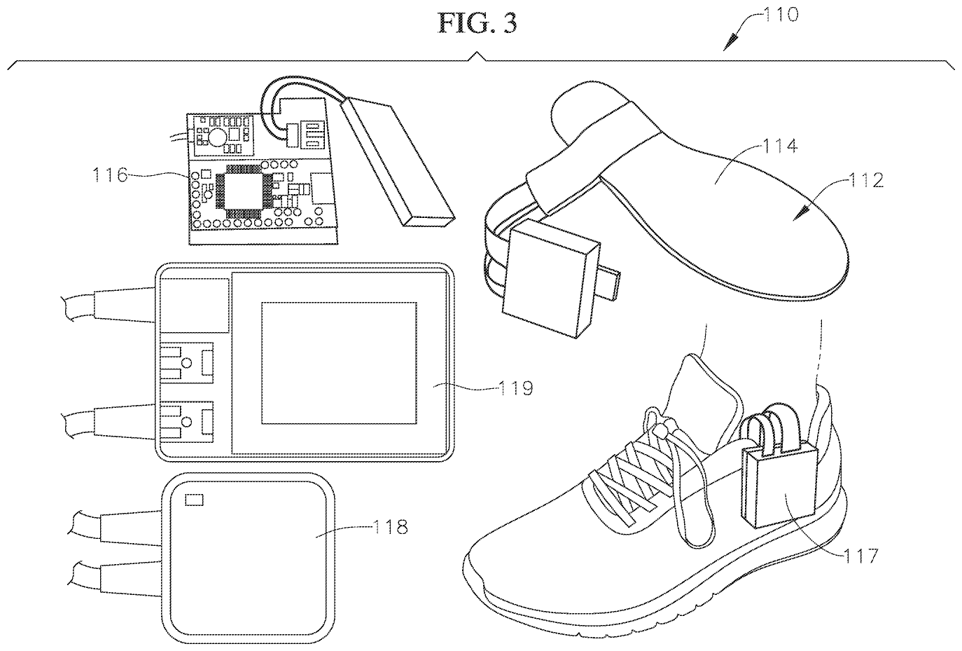

It has been demonstrated that support vector regression (SVR) models can be used to extract accurate estimates of fundamental gait parameters (i.e., stride length, velocity, and foot clearance), from a custom-engineered instrumented insole 110 (such as the “SportSole” system shown in FIG. 3) during walking and running tasks. The “SportSole” system comprises two insole modules and a data logger. Additionally, these learning-based models are robust to inter-subject variability, thereby making it unnecessary to collect subject-specific training data.

The insole module 110 includes a logic unit 116 (see FIG. 3), which is housed in a custom plastic enclosure 117, and a multi-cell piezoresistive sensor 112 with embedded IMU 114 (see FIG. 3). The data logger consists of a single-board computer (not shown) and a small Wi-Fi router 118 (see FIG. 3). The instrumented insole 110 can be fitted inside regular sneakers, and the logic unit 116 can be housed inside a customized 3D-printed enclosure and secured to the postero-lateral side of the user's sneakers with a plastic clip (see FIG. 3). Both sensors are sandwiched between layers of abrasion-resistant foam. Data measured by each insole are sampled at 500 Hz and sent through UDP over WLAN to the single-board computer running the data logger software. Along with a wireless connection module, this insole module 110 is also responsible for synchronizing the overall system through Reference Time Broadcast Synchronization.

A novel 2-step calibration method to improve the estimates of stride-to-stride gait parameters obtained with wearable sensors was developed. The first step relies on conventional methods (i.e., ZUPT and VDC), and the second step entails using inertial features and test subjects' anthropometric characteristics to improve the estimates obtained with the first step through regression models.

This work systematically explored reliability and validity of these learning-based models, both in walking and running tasks. Rather than replacing conventional data processing techniques, the learning-based methods proposed improved the stride-to-stride gait parameters computed using conventional methods by reducing systematic errors. This approach generates accurate estimates without the computational burden that is typically associated with end-to-end machine learning models that take raw sensor data as inputs.

Moreover, a simple yet effective heuristic method to select input features and tune SVR models was proposed. Analysis of the feature selection ratios confirmed the importance of the raw estimates obtained with ZUPT and VDC in determining all gait parameters, both for walking and for running tasks. A larger number of additional features were required to produce the optimal GN-SVR models in the running task compared to the walking task, thereby suggesting that these raw estimates are less reliable in highly dynamic tasks.

It will be understood that the embodiments described hereinabove are merely exemplary and that a person skilled in the art may make many variations and modifications without departing from the spirit and scope of the present invention. It is worth noting that, even though the proposed inference models were validated on a labelled dataset collected using a custom-engineered instrumented footwear, these methods can be readily applied to any IMU-based footwear system, such as instrumented insoles and on-shoe sensors. All such variations and modifications are intended to be included within the scope of the invention.

What is claimed is:

1. A method for creating an individualized machine learning inference model to augment validity and reliability of a wearable motion capture device, comprising the steps of:
providing a user with a wearable motion capture device;
acquiring measurements using said wearable motion capture device;
computing a first estimate of one or more gait parameters using said measurements;
accessing a database containing previously collected observations of gait data from said user or from other users, using said wearable device and a more accurate reference device;
identifying an optimal set of input features and parameters using a subset of said previously collected observations of gait data from said wearable device that is most informative for said user; and
developing an individualized machine learning inference model using said optimal set of input features and parameters from said subset of previously collected observations of gait data from said wearable device and said reference device.

2. The method of claim 1, further comprising the step of applying said individualized machine learning inference model to said measurements to obtain a second estimate of one or more gait parameters.

3. The method of claim 1, wherein said one or more gait parameters of said first estimate are the same as said one or more gait parameters of said second estimate.

4. The method of claim 1, wherein said one or more gait parameters of said first estimate are different from said one or more gait parameters of said second estimate.

5. The method of claim 1, wherein said measurements involve center of pressure and/or dynamic margin of stability.

6. The method of claim 1, wherein said measurements involve inter-limb parameters.

7. The method of claim 1, wherein said one or more gait parameters of said first estimate are selected from the group consisting of stride length, foot-ground clearance, foot trajectory, cadence, double support time, single support time, walking or running speed, center of pressure, stride width, and margin of stability.

8. The method of claim 1, further comprising the step of generating dynamic plantar pressure maps and/or center of pressure trajectories based on said measurements.

9. The method of claim 1, further comprising the step of classifying activities of daily living based on said measurements.

10. The method of claim 1, wherein said method is implemented by a mobile device having GPS in order to realize a portable navigation system.

11. The method of claim 1, wherein walking and/or balance exercises are monitored and/or administered, either remotely or in person, using said measurements.

12. The method of claim 1, further comprising the step of providing gait and/or balance rehabilitation to said user.

13. The method of claim 1, further comprising the use of said individualized machine learning inference model to diagnose medical conditions affecting human gait and balance, or predict the risk of musculoskeletal injuries.

14. The method of claim 1, wherein said method is performed by one or more single-board computers running a Linux distribution with a real-time kernel operating in headless mode.

15. The method of claim 14, wherein each single-board computer uses at least one wireless connection module to synchronize said measurements from multiple wireless sensors, each single-board computer also being configured to write said measurements to a micro-SD card.

16. The method of claim 1, wherein said individualized machine learning inference model involves one or more of the techniques from the group consisting of: Support Vector Regression; Gaussian Mixture Models; Gaussian Process Regression; and Support Vector Machines.

17. The method of claim 1, wherein said first estimate of one or more gait parameters is obtained by using conventional data processing techniques to obtain spatiotemporal, kinematic or kinetic gait parameters.

18. The method of claim 1, wherein said individualized machine learning inference model is adapted to be implemented through a cloud service or a mobile device.

19. A gait measurement system, comprising:
at least one insole module for placement in a shoe of a user, said at least one insole module including a piezoresistive sensor, an inertial sensor, a logic unit communicatively coupled to said piezoresistive sensor and to said inertial sensor, and a transmission unit;
a database communicatively coupled to said at least one insole module, said database containing previously collected observations of gait data; and
a computing unit communicatively coupled to said inertial sensor and said piezoresistive sensor via said transmission unit, wherein said computing unit is configured to receive measurements from said at least one insole module and to train and implement an individualized support vector regression model by computing a first estimate of one or more gait parameters using said measurements, comparing said first estimate of one or more gait parameters to said previously collected observations of gait data, using said first estimate of one or more gait parameters to identify a subset of said previously collected observations of gait data that is most informative for said user, developing an individualized machine learning inference model using said subset of said previously collected observations of gait data, and applying said individualized machine learning inference model to said measurements to obtain a second estimate of said one or more gait parameters.

20. The gait measurement system of claim 19, wherein said one or more gait parameters of said first estimate are the same as said one or more gait parameters of said second estimate.

21. The gait measurement system of claim 19, wherein said one or more gait parameters of said first estimate are different from said one or more gait parameters of said second estimate.

22. The gait measurement system of claim 19, wherein said system is configured to estimate center of pressure and/or dynamic margin of stability.

23. The gait measurement system of claim 19, wherein said system is adapted to measure inter-limb parameters.

24. The gait measurement system of claim 19, wherein said system is adapted to measure one or more gait parameters selected from the group consisting of stride length, foot-ground clearance, foot trajectory, cadence, double support time, single support time, walking speed, center of pressure, and margin of stability.

25. The gait measurement system of claim 24, wherein said computing unit is further adapted to generate dynamic plantar pressure maps and/or center of pressure trajectories.

26. The gait measurement system of claim 19, wherein said computing unit is adapted to classify activities of daily living, with or without integrating additional inputs from sensors embedded in off-the-shelf mobile devices such as a mobile phone and a wrist-worn device.

27. The gait measurement system of claim 19, wherein said system is adapted to cooperate with a mobile device having GPS in order to realize a portable navigation system.

28. The gait measurement system of claim 19, wherein said system is adapted to remotely monitor and administer walking and/or balance exercises.

29. The gait measurement system of claim 19, wherein said system is adapted to provide gait and/or balance rehabilitation or for diagnostic purposes.

30. The gait measurement system of claim 19, wherein said computing unit comprises a single-board computer running a Linux distribution with a real-time kernel operating in headless mode.

31. The gait measurement system of claim 19, wherein said computing unit is configured to synchronize said measurements and to write said measurements to a micro-SD card.

32. The gait measurement system of claim 19, wherein said second estimate of one or more gait parameters is obtained using a method selected from the group consisting of: Support Vector Regression; Gaussian Mixture Models; Gaussian Process Regression; and Support Vector Machines.

33. The gait measurement system of claim 19, wherein said first estimate of one or more gait parameters is obtained by using conventional data processing techniques to obtain spatiotemporal, kinematic or kinetic gait parameters.

34. The gait measurement system of claim 19, wherein said system is configured to use said individualized machine learning inference via a cloud service or a mobile device.
